(19) United States
(12) Patent Application Publication
(10) Pub. No.: US 2013/0053599 A1
(43) Pub. Date: Feb. 28, 2013

(54) CATALYSTS FOR PRODUCING ACRYLIC ACIDS AND ACRYLATES

(71) Applicants: Heiko Weiner, Pasadena, TX (US); Josefina T. Chapman, Houston, TX (US); Alexandra S. Locke, Salt Lake City, UT (US); Dick Nagaki, The Woodlands, TX (US); Craig J. Peterson, Houston, TX (US); Mark O. Scates, Houston, TX (US)

(72) Inventors: Heiko Weiner, Pasadena, TX (US); Josefina T. Chapman, Houston, TX (US); Alexandra S. Locke, Salt Lake City, UT (US); Dick Nagaki, The Woodlands, TX (US); Craig J. Peterson, Houston, TX (US); Mark O. Scates, Houston, TX (US)

(73) Assignee: CELANESE INTERNATIONAL CORPORATION, Dallas, TX (US)

(21) Appl. No.: 13/214,325

(22) Filed: Aug. 22, 2011

(51) Int. Cl. B01J 27/14; C07C 67/30; B01J 29/04; C07C 51/347; B01J 27/198; B01J 27/182

(52) U.S. Cl. 560/211; 502/209; 502/208; 502/214; 502/87; 562/599

(57) ABSTRACT

In one embodiment, the invention is to a catalyst composition comprising titanium, phosphorus, and less than 1 wt. % vanadium. The catalyst composition has a molar ratio of phosphorus to titanium of at least 1.0:1.0.

### FIELD OF THE INVENTION

The present invention relates generally to the production of acrylic acid. More specifically, the present invention relates to catalysts used in the production of acrylic acid and acrylates via the condensation of acetic acid and formaldehyde.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes the reaction of acetylene with water and carbon monoxide or the reaction of an alcohol and carbon monoxide to yield the acrylate ester. Another conventional process involves the reaction of ketene (often obtained by the pyrolysis of acetone or acetic acid) with formaldehyde. These processes have become obsolete for economic, environmental, or other reasons.

Another acrylic acid and acrylates production method utilizes the condensation of formaldehyde and acetic acid and/or carboxylic acid esters. This reaction is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). The acetic acid conversions in these reactions, however, may leave room for improvement.

Thus, the need exists for improved processes for producing acrylic acid and acrylates, and for improved catalysts capable of providing high acetic acid conversions in the formation of acrylic acid.

### SUMMARY OF THE INVENTION

In one embodiment, the invention is to a catalyst composition comprising from 18 wt. % to 35 wt. % phosphorus, from 11 wt. % to 39 wt. % titanium, and less than 1 wt. % vanadium. In one embodiment, the molar ratio of phosphorus to titanium in the catalyst composition is at least 1:1. In some preferred embodiments, the inventive catalyst is substantially free of vanadium.

In another embodiment, the present invention is to a process for producing the above-mentioned catalyst composition. The process comprises the steps of contacting a titanium precursor mixture with phosphoric acid to form a catalyst precursor mixture and calcining the catalyst precursor mixture to form the catalyst composition. In one embodiment, the titanium precursor mixture and the phosphoric acid are contacted under condition effective to produce the catalyst composition having a molar ratio of phosphorus to titanium of at least 1:1.

In another embodiment, the present invention is to a process for producing acrylic acid. The process comprises the step of contacting an alkanoic acid and an alkylenating agent over the above-identified catalyst under conditions effective to produce acrylic acid and/or acrylate.

### DETAILED DESCRIPTION OF THE INVENTION

### Introduction

Production of unsaturated carboxylic acids such as acrylic acid and methacrylic acid and the ester derivatives thereof via most conventional processes have been limited by economic and environmental constraints. One process for producing these acids and esters involves the aldol condensation of formaldehyde and (i) acetic acid and/or (ii) ethyl acetate over a catalyst. Exemplary classes of conventional catalysts for this reaction include binary vanadium-titanium phosphates, vanadium-silica-phosphates, and alkali metal-promoted silicas, e.g., cesium- or potassium-promoted silicas. The alkali metal-promoted silicas, however, have been known to exhibit only low to moderate activity when used in aldol condensation reactions. As a result, the alkali metal-promoted silicas typically require metal dopants, e.g., bismuth, lanthanum, lead, thallium, and tungsten, to improve catalyst performance.

Binary vanadium-titanium phosphates have been studied with regard to the condensation of acetic acid and formaldehyde (or a methanol/oxygen mixture) to form acrylic acid. Catalysts with a vanadium:titanium:phosphorus molar ratio of 1:2:x, where x is varied from 4.0 to 7.0, have traditionally shown that the catalyst activity decreases steadily as the phosphorus content increased (see, for example M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987), discussed above). Although these catalysts may yield some aldol condensation products, e.g., acrylic acid and methyl acrylate, the conversions and selectivities are lower than desired.

### Catalyst Composition

It has now been discovered that certain catalysts effectively catalyze the aldol condensation of a carboxylic acid with an alkylenating agent, e.g. a methylenating agent, such as formaldehyde to form an unsaturated acid. Preferably, the reaction is an aldol condensation reaction of acetic acid with formaldehyde to form acrylic acid. In one embodiment, the present invention is to a catalyst composition comprising titanium, phosphorus and, optionally, oxygen. Preferably, the molar ratio of phosphorus to titanium, e.g., the “phosphorus-titanium ratio,” in an active phase of the catalyst composition is greater than 1:1, e.g., greater than 1.5:1, greater than 2:1, or greater than 2.5:1. The active phase is the portion of the catalyst that comprises the components that promote the catalysis. In terms of ranges, the phosphorus-titanium ratio in the active phase of the catalyst composition may range from 1 to 3, e.g., from 1 to 3; from 1.66 to2.5 or from 2.0 to 2.2.

In some embodiments, the inventive catalyst composition comprises only small amounts of vanadium, for example, the catalyst comprises less than 1 wt. % vanadium, e.g., less than 0.5 wt. % or less than 0.1 wt. %. Preferably, the inventive catalyst composition is substantially free of vanadium, e.g., the catalyst composition comprises less than 0.1 wt. % vanadium. In a preferred embodiment, the catalyst composition comprises no vanadium, e.g., the catalyst composition is vanadium free. In terms of ranges, the catalyst composition may comprise from 0 wt. % to 1 wt. % vanadium, e.g., from 0 wt. % to 0.5 wt. % or from 0.05 wt. % to 0.1 wt. %. In one particular embodiment, the inventive composition comprises from 18 wt. % to 35 wt. % phosphorus; from 11 wt. % to 39 wt. % titanium, and less than 1 wt. % vanadium.

The total amounts of titanium and phosphorus in the catalyst compositions of the invention may vary widely so long as these components are present in the above-described ranges. In some embodiments, for example, the catalyst comprises at least 11 wt. % titanium, e.g., at least 15 wt. % or at least 20 wt. %. In terms of ranges, the catalyst may comprise from 10 wt. % to 40 wt. % titanium, e.g., from 11 wt. % to 39 wt. %, or from 15.5 wt. % to 35.5 wt. % titanium. In some embodiments, the catalyst comprises at least 18 wt. % phosphorus, e.g., at least 23 wt. % or at least 28 wt. %. In terms of ranges, the catalyst may comprise from 15 wt. % to 40 wt. % phosphorus, e.g., from 18 wt. % to 35 wt. % or from 23 wt. % to 30 wt. %. The catalyst composition may optionally comprise oxygen. In such cases, the catalyst may comprise at least 30 wt. % oxygen, e.g., at least 35 wt. % or at least 50 wt. %. In terms of ranges, the catalyst may comprise from 30 wt. % to 65 wt. % oxygen, e.g., from 35 wt. % to 60 wt. % or from 40 wt. % to 55 wt. %.

In some embodiments, the titanium is present in compound form such as in the form of titanium dioxide. For example, the catalyst may comprise titanium dioxide in an amount ranging from 0.1 wt. % to 95 wt. %, e.g., from 5 wt. % to 50 wt. % or from 7 wt. % to 25 wt. %. In these cases, the titanium dioxide may be in the rutile and/or anatase form, with the anatase form being preferred. If present, the catalyst preferably comprises at least 5 wt. % anatase titanium dioxide, e.g., at least 10 wt. % anatase titanium dioxide, or at least 50 wt. % anatase titanium dioxide. Preferably less than 20 wt. % of the titanium dioxide, if present in the catalyst, is in rutile form, e.g., less than 10 wt. % or less than 5 wt. %. In other embodiments, the catalyst comprises anatase titanium dioxide in an amount of at least 5 wt. %, e.g., at least 10 wt. % or at least 20 wt. %. In another embodiment, the titanium is present in the form of amorphous titanium hydroxide gel, which is preferably converted to an amorphous titanium phosphate such as TiP₂O₇.

The titanium hydroxide gel may be prepared by any suitable means including, but not limited to, the hydrolysis of titanium alkoxides, substituted titanium alkoxides, or titanium halides. In other embodiments, colloidal titania sols and/or dispersions may be employed. In one embodiment, titania coated colloidal particles or supports are used as a source of titanium dioxide. The hydrous titania may be amorphous or may contain portions of anatase and/or rutile depending on preparation method and heat treatment.

Upon treatment with a phosphating agent, the various forms of titania may be converted to titanium phosphates and/or titanium pyrophosphates. In some cases, a portion of the titanium may be present as unconverted titania and, hence, will be present in the final catalyst as anatase or rutile forms.

Generally speaking, the proportion of the crystalline forms of titania present in the catalyst is dependent on the titanium precursor, the preparative method, and/or the post-phosphorylating treatment. In one embodiment, the amount of anatase and rutile present in the active phase of the catalyst is minimized. The amount of crystalline titania, however, may be high with only a thin shell of porous catalyst existing on the titania support.

The inventive catalyst composition may, in some embodiments, further comprise a support. Preferably, the support is selected from the group consisting of silica, alumina, zirconia, titania, aluminosilicates, zeolitic materials, and mixtures thereof.

In one embodiment, suitable phosphorus compounds that serve as a source of phosphorus in the catalyst contain pentavalent phosphorus and include, but are not limited to, phosphoric acid, ammonium phosphates, phosphorus pentoxide, polyphosphoric acid, or phosphorus perhalides such as phosphorus pentachloride, and mixtures thereof.

Preferably, the active phase of the catalyst corresponds to the formula

TiₐP_{b}O_{c}

wherein the letters a, b, and c are the relative molar amounts (relative to 1.0) of phosphorus, titanium, and oxygen, respectively, in the catalyst. In these embodiments, the ratio of b to a is preferably greater than 1.0:1.0, e.g., greater than 1.66:1.0, greater than 2.0:1.0, or greater than 2.1:1.0. Preferred ranges for molar variables a, b, and c are shown in Table 1.

TABLE 1
Molar Ranges

| | Molar Range | Molar Range | Molar Range |
|---|---|---|---|
| a | 1 | 1 | 1 |
| b | 1 to 3 | 1.5 to 2.5 | 2.0 to 2.3 |
| c | 2 to 10.5 | 2.25 to 9 | 3 to 8 |

In some embodiments, the catalyst further comprises additional metals. These additional metals may function as promoters. If present, the additional metals may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

If the catalyst comprises additional metal(s) and/or metal oxides(s), the catalyst optionally may comprise additional metals and/or metal oxides in an amount from 0.001 wt. % to 30 wt. %, e.g., from 0.01 wt. % to 5 wt. % or from 0.1 wt. % to 5 wt. %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt. % to 99.9 wt. %, e.g., from 78 wt. % to 97 wt. % or from 80 wt. % to 95 wt. %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

In some embodiments, the support may be a high surface area support, e.g., a support having a surface area of at least 1 m²/g, e.g., at least 20 m²/g or at least 50 m²/g, as determined by BET measurements. The catalyst support may include pores, optionally having an average pore diameter ranging from 5 nm to 200 nm, e.g., from 5 nm to 50 nm or from 10 nm to 25 nm. The catalyst optionally has an average pore volume of from 0.05 cm³/g to 3 cm³/g, e.g., from 0.05 cm³/g to 0.1 cm³/g or from 0.08 cm³/g to 0.1 cm³/g, as determined by BET measurements. Preferably, at least 50% of the pore volume or surface area, e.g., at least 70% or at least 80%, is provided by pores having the diameters discussed above. Pores may be formed and/or modified by pore modification agents, which are discussed below. In another embodiment, the ratio of microporosity to macroporosity ranges from 95:5 to 85:15, e.g., from 75:25 to 70:30. Microporosity refers to pores smaller than 2 nm in diameter, and movement in micropores may be described by activated diffusion. Mesoporosity refers to pores greater than 2 nm and less than 50 nm is diameter. Flow through mesopores may be described by Knudson diffusion. Macroporosity refers to pores greater than 50 nm in diameter and flow though macropores may be described by bulk diffusion. Thus, in some embodiments, it is desirable to balance the surface area, pore size distribution, catalyst or support particle size and shape, and rates of reaction with the rate of diffusion of the reactant and products in and out of the pores to optimize catalytic performance.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In one embodiment, the inventive catalyst composition comprises a pore modification agent. A preferred type of pore modification agent is thermally stable and has a substantial vapor pressure at a temperature below 300° C., e.g., below 250° C. In one embodiment, the pore modification agent has a vapor pressure of at least 0.1 kPa, e.g., at least 0.5 kPa, at a temperature between about 150° C. and about 250° C., e.g., between about 150° C. and about 200° C.

In some embodiments, the pore modification agent has a relatively high melting point, e.g., greater than 60° C., e.g., greater than 75° C., so that it does not melt during compression of the catalyst precursor into a slug, tablet, or pellet. Preferably, the pore modification agent comprises a relatively pure material rather than a mixture. As such, lower melting components will not liquefy under compression during formation of slugs or tablets. For example, where the pore modification agent is a fatty acid, lower melting components of the fatty acid mixtures may be removed as liquids by pressing. If this phenomenon occurs during slug or tablet compression, the flow of liquid may disturb the pore structure and produce an undesirable distribution of pore volume as a function of pore diameter on the catalyst composition. In other embodiments, the pore modification agents have a significant vapor pressure at temperatures below their melting points, so that they can be removed by sublimination into a carrier gas.

For example, the pore modification agent may be a fatty acid corresponding to the formula CH₃(CH₂)ₓCOOH where x>8. Exemplary fatty acids include stearic acid (x=16), palmitic acid (x=14), lauric acid (x=10), myristic acid (x=12). The esters of these acids and amides or other functionalized forms of such acids, for example, stearamide (CH₃(CH₂)₁₆CONH₂) may also be used. Suitable esters may include methyl esters as well as glycerides such as stearin (glycerol tristearate). Mixtures of fatty acids may be used, but substantially pure acids, particularly stearic acid, are generally preferred over mixtures.

Other preferred pore modification agents include but are not limited to polynuclear organic compounds such as naphthalene, graphite, natural burnout components such as cellulose and its cellulosic derivatives, starches, natural and synthetic oligomers and polymers such as polyvinyl alcohols and polyacrylic acids and esters.

### Catalyst Preparation

In an embodiment of the present invention, the catalyst composition is formed by a process comprising the step of preparing a titanium precursor mixture. In one embodiment, the titanium precursor mixture is prepared by contacting a titanium precursor, e.g., titanium pyrophosphate, with an alcohol, such as 2-propanol, ethanol, or i-butanol. This step may result in a diluted titanium precursor mixture, which may be slowly added to a colloidal silica, which may include colloidal silica, optionally in water. Phosphoric acid is then slowly added to the titanium precursor mixture to form the catalyst precursor mixture. The process further comprises the step of drying the catalyst precursor mixture and/or calcining the catalyst composition, for example, as described below.

In preferred embodiments, the titanium precursor is selected from a group consisting of Ti(OR)₄, LₓTi(OR)_{y} complexes, TiCl_{z}, hydrated titania sols and colloidal TiO₂, wherein R=methyl, ethyl, propyl, and butyl; L=acetylacetone, or similar bidentate ligands; x=1-3; y=1-3; and z=3-4. Most preferably, the titanium precursor comprises TiP₂O₇ and/or Ti(OiPr)₄.

In some embodiments, e.g., embodiments where the catalyst is unsupported, the catalyst may be formed by a process comprising the step of dissolving at least one oxide additive and an acid, e.g., phosphoric acid, optionally in water, to form an additive solution comprising at least 0.04 wt. % oxide additive, e.g., at least 0.1 wt. % or at least 1 wt. %. The process may further comprise the steps of adding a titanium precursor to the additive solution to form a catalyst precursor mixture and drying the catalyst precursor mixture to form the catalyst composition.

In preferred embodiments, where the catalyst is unsupported, the catalyst composition may be formed via a process comprising the step of dissolving at least one oxide additive and an acid, e.g., phosphoric acid, in water to form an additive solution comprising at least 0.04 wt. % oxide additive, e.g., at least 0.1 wt. % or at least 1 wt. %. The process further comprises the steps of contacting the additive solution with a titanium precursor, e.g., hydrous titania or Ti(OiPr)₄, to form a titania sol or gel. Preferably, the process further comprises the step of drying the wet catalyst precursor to form a dried catalyst composition and optionally, further calcining the dried catalyst composition. The amounts of the titanium precursor is determined such that the resultant dried catalyst composition has a molar ratio of phosphorus to titanium greater than 1:1, e.g., greater than 1.25:1, greater than 1.5:1, or greater than 2:1.

The process, in one embodiment, may further comprise calcining the dried catalyst, which, preferably, is conducted in accordance with a temperature profile. As one example, the temperature profile comprises an increasing stair step temperature profile comprising a plurality of increasing hold temperatures. The temperature increases at a rate from 1° C. to 5° C. per minute between said hold temperatures. Preferably, the hold temperatures comprise a first, second, third, and fourth hold temperature. The first hold temperature may range from 150° C. and 300° C., e.g., from 175° C. and 275° C., preferably being about 160° C. The second hold temperature may range from 250° C. and 500° C., e.g., from 300° C. and 400° C., preferably being about 250° C. The third hold temperature may range from 300° C. and 700° C., e.g., from 450° C. and 650° C., preferably being about 300° C. The fourth hold temperature may range from 400° C. and 700° C., e.g., from 450° C. and 650° C., preferably being about 450° C. Of course, other temperature profiles may be suitable. The calcination of the mixture may be done in an inert atmosphere, air or an oxygen-containing gas at the desired temperatures. Steam, a hydrocarbon or other gases or vapors may be added to the atmosphere during the calcination step or post-calcination to cause desired effects on physical and chemical surface properties as well as textural properties such as increase macroporosity.

In one preferred embodiment, the temperature profile comprises:

i) heating the dried catalyst from room temperature to 160° C. at a rate of 10° C. per minute;

ii) heating the dried catalyst composition at 160° C. for 2 hours;

iii) heating the dried catalyst composition from 160° C. to 250° C. at a rate of 3° C. per minute;

iv) heating the dried catalyst composition at 250° C. for 2 hours;

v) heating the dried catalyst composition from 250° C. to 300° C. at a rate of 3° C. per minute;

vi) heating the dried catalyst composition at 300° C. for 6 hours;

vii) heating the dried catalyst composition from 300° C. to 450° C. at a rate of 3° C. per minute; and

viii) heating the dried catalyst composition at 450° C. for 2 hours.

In another embodiment, the temperature profile comprises:

i) contacting the catalyst composition with flowing air at a first temperature; ii) contacting the catalyst composition with flowing air at a second temperature greater than the first temperature; and

iii) contacting the catalyst composition with static air at a third temperature greater than the first and second temperatures.

The first hold temperature may range from 110° C. and 210° C., e.g., from 135° C. and 185° C., preferably being about 160° C. The second hold temperature may range from 300° C. and 400° C., e.g., from 325° C. and 375° C., preferably being about 350° C. The third hold temperature may range from 400° C. and 500° C., e.g., from 425° C. and 475° C., preferably being about 450° C. In one embodiment, a first drying stage uses flowing air at 160° C. for approximately 2 hours; a second drying stage uses flowing air at 350° C. for approximately 4 hours, and a third drying stage uses static air at 450° C. for eight hours. Of course, other temperature profiles may be suitable.

In embodiments where the catalyst is supported, the catalyst compositions are formed through metal impregnation of a support (optionally modified support), although other processes such as chemical vapor deposition may also be employed.

In one embodiment, the catalysts are made by impregnating the support, with a solution of the metals or salts thereof in a suitable solvent, followed by drying and optional calcination. Solutions of the modifiers or additives may also be impregnated onto the support in a similar manner. The impregnation and drying procedure may be repeated more than once in order to achieve the desired loading of metals, modifiers, and/or other additives. In some cases, there may be competition between the modifier and the metal for active sites on the support. Accordingly, it may be desirable for the modifier to be incorporated before the metal. Multiple impregnation steps with aqueous solutions may to reduce the strength of the catalyst particles if the particles are fully dried between impregnation steps. Thus, it is preferable to allow some moisture to be retained in the catalyst between successive impregnations. In one embodiment, when using non-aqueous solutions, the modifier and/or additive are introduced first by one or more impregnations with a suitable non-aqueous solution, e.g., a solution of an alkoxide or acetate of the modifier metal in an alcohol, e.g., ethanol, followed by drying. The metal may then be incorporated by a similar procedure using a suitable solution of a metal compound.

In other embodiments, the modifier is incorporated into the composition by co-gelling or co-precipitating a compound of the modifier element with the silica, or by hydrolysis of a mixture of the modifier element halide with a silicon halide. Methods of preparing mixed oxides of silica and zirconia by sol gel processing are described by Bosman, et al., in J Catalysis, Vol. 148, (1994), page 660 and by Monros et al., in J Materials Science, Vol. 28, (1993), page 5832. Also, doping of silica spheres with boron during gelation from tetraethyl orthosilicate (TEOS) is described by Jubb and Bowen in J Material Science, Vol. 22, (1987), pages 1963-1970. Methods of preparing porous silicas are described in Iler R K, The Chemistry of Silica, (Wiley, New York, 1979), and in Brinker C J & Scherer G W Sol-Gel Science published by Academic Press (1990).

The catalyst composition, in some embodiments, will be used in a fixed bed reactor for forming the desired product, e.g., acrylic acid or alkyl acrylate. Thus, the catalyst is preferably formed into shaped units, e.g., spheres, granules, pellets, powders, aggregates, or extrudates, typically having maximum and minimum dimensions in the range of 1 to 25 mm, e.g., from 2 to 15 mm. Where an impregnation technique is employed, the support may be shaped prior to impregnation. Alternatively, the composition may be shaped at any suitable stage in the production of the catalyst. The catalyst also may be effective in other forms, e.g. powders or small beads and may be used in these forms. In one embodiment, the catalyst is used in a fluidized bed reactor. In this case, the catalyst may be prepared via spray drying or spray thermal decomposition. Preferably, the resultant catalyst has a particle size of greater than 300 microns, e.g., greater than 500 microns.

### Production of Acrylic Acid and Acrylate Esters

In other embodiments, the invention is to a process for producing unsaturated acids, e.g., acrylic acids, or esters thereof (alkyl acrylates), by contacting an alkanoic acid with an alkylenating agent, e.g., a methylenating agent, under conditions effective to produce the unsaturated acid and/or acrylate. Preferably, acetic acid is reacted with formaldehyde in the presence of the inventive catalyst composition. The alkanoic acid, or ester of an alkanoic acid, may be of the formula R′—CH₂—COOR, where R and R′ are each, independently, hydrogen or a saturated or unsaturated alkyl or aryl group. As and example, R and R′ may be a lower alkyl group containing for example 1-4 carbon atoms. In one embodiment, an alkanoic acid anhydride may be used as the source of the alkanoic acid. In one embodiment, the reaction is conducted in the presence of an alcohol, preferably the alcohol that corresponds to the desired ester, e.g., methanol. In addition to reactions used in the production of acrylic acid, the inventive catalyst, in other embodiments, may be employed to catalyze other reactions. Examples of these other reactions include, but are not limited to butane oxidation to maleic anhydride, acrolein production from formaldehyde and acetaldehyde, and methacrylic acid production from formaldehyde and propionic acid.

The acetic acid may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syngas”) that is derived from any available carbon source. U.S. Pat. No. 6,232,352, which is hereby incorporated by reference, for example, teaches a method of retrofitting a methanol plant for the manufacture of acetic acid. By retrofitting a methanol plant, the large capital costs associated with carbon monoxide generation for a new acetic acid plant are significantly reduced or largely eliminated. All or part of the syngas is diverted from the methanol synthesis loop and supplied to a separator unit to recover carbon monoxide and hydrogen, which are then used to produce acetic acid.

Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, all of which are hereby incorporated by reference.

U.S. Pat. No. RE 35,377, which is hereby incorporated by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. U.S. Pat. No. 5,821,111, which discloses a process for converting waste biomass through gasification into syngas, as well as U.S. Pat. No. 6,685,754 are hereby incorporated by reference.

In one optional embodiment, the acetic acid that is utilized in the condensation reaction comprises acetic acid and may also comprise other carboxylic acids, e.g., propionic acid, esters, and anhydrides, as well as acetaldehyde and acetone. In one embodiment, the acetic acid fed to the hydrogenation reaction comprises propionic acid. For example the propionic acid in the acetic acid feed stream may range from 0.001 wt. % to 15 wt. %, e.g., from 0.001 wt. % to 0.11 wt. %, from 0.125 wt. % to 12.5 wt. %, from 1.25 wt. % to 11.25, or from 3.75 wt. % to 8.75 wt. %. Thus, the acetic acid feed stream may be a cruder acetic acid feed stream , e.g., a less-refined acetic acid feed stream.

As used herein, “alkylenating agent” means an aldehyde or precursor to an aldehyde suitable for reacting with the alkanoic acid, e.g., acetic acid, in an aldol condensation reaction to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, and butanal.

The alkylenating agent, e.g., formaldehyde, may be added from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a formox unit, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 350° C. or over an acid catalyst at over 100° C. to form the formaldehyde.

In one embodiment, the alkylenating agent corresponds to Formula I.

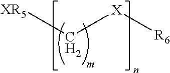

In this formula, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1 dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1, 1 dimethoxymethane; higher formals of formaldehyde and methanol; and CH₃—O—(CH₂—O)ᵢ—CH₃ where i is 2.

The alkylenating agent may be used with or without an organic or inorganic solvent.

The term “formalin,” refers to a mixture of formaldehyde, methanol, and water. In one embodiment, formalin comprises from 25 wt. % to 65 wt. % formaldehyde; from 0.01 wt. % to 25 wt. % methanol; and from 25 wt. % to 70 wt. % water. In cases where a mixture of formaldehyde, methanol, and methyl propionate is used, the mixture comprises less than 10 wt. % water, e.g., less than 5 wt. % or less than 1 wt. %.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a mole percentage based on acetic acid in the feed. The conversion of acetic acid may be at least 11 mol. %, e.g., at least 20 mol. %, at least 40 mol. %, or at least 50 mol. %. In another embodiment, the reaction may be conducted wherein the molar ratio of acetic acid to alkylenating agent is at least 0.55:1, e.g., at least 1:1.

Selectivity is expressed as the ratio of the amount of carbon in the desired product(s) and the amount of carbon in the total products. This ratio may be multiplied by 100 to arrive at the selectivity. Preferably, the catalyst selectivity to acrylates, e.g., acrylic acid and methyl acrylate, is at least 40 mol. %, e.g., at least 50 mol. %, at least 60 mol. %, or at least 70 mol. %. In some embodiments, the selectivity to acrylic acid is at least 30 mol. %, e.g., at least 40 mol. %, or at least 50 mol. %; and/or the selectivity to methyl acrylate is at least 10 mol. %, e.g., at least 15 mol. %, or at least 20 mol. %.

The term “productivity,” as used herein, refers to the grams of a specified product, e.g., acrylates, formed during the condensation based on the liters of catalyst used per hour. A productivity of at least 20 grams of acrylates per liter catalyst per hour, e.g., at least 40 grams of acrylates per liter catalyst per hour or at least 100 grams of acrylates per liter catalyst per hour, is preferred. In terms of ranges, the productivity preferably is from 20 to 500 grams of acrylates per liter catalyst per hour, e.g., from 20 to 200 grams of acrylates per kilogram catalyst per hour or from 40 to 140 grams of acrylates per kilogram catalyst per hour.

As noted above, the inventive catalyst compositions provide for high conversions of acetic acid. Advantageously, these high conversions are achieved while maintaining selectivity to the desired acrylates, e.g., acrylic acid and/or methyl acrylate. As a result, acrylate productivity is improved, as compared to conventional productivity with conventional catalysts.

The acetic acid conversion, in some embodiments, may vary depending upon the reaction temperature. In one embodiment, for example, when the reaction temperature is approximately 340° C., the acetic acid conversion is at least 11%, e.g., at least 15% or at least 25%. The selectivity to acrylates is maintained at, for example, at least 60%, e.g., at least 65%, at least 75% or at least 90%. Accordingly, the productivity, e.g., the space time yield, of acrylates is at least 29 grams per liter catalyst per hour, e.g., at least 40 grams per liter or at least 55 grams per liter, when the reaction temperature is approximately 340° C.

In another embodiment where the reaction temperature is approximately 350° C., the acetic acid conversion is at least 28%, e.g., at least 30% or at least 35%. The selectivity to acrylates is maintained at, for example, at least 60%, e.g., at least 65%, at least 75% or at least 90%. Accordingly, the productivity, e.g., the space time yield, of acrylates is at least 57 grams per liter of catalyst per hour, e.g., at least 70 grams per liter of catalyst per hour or at least 85 grams per liter of catalyst per hour, when the reaction temperature is approximately 355° C.

In another embodiment where the reaction temperature is approximately 370° C., the acetic acid conversion is at least 38%, e.g., at least 40% or at least 45%. The selectivity to acrylates is maintained at, for example, at least 60%, e.g., at least 65%, at least 75% or at least 90%. Accordingly, the productivity, e.g., the space time yield, of acrylates is at least 97 grams per liter of catalyst per hour, e.g., at least 110 grams per liter of catalyst per hour or at least 125 grams per liter of catalyst per hour, when the reaction temperature is approximately 370° C.

It has now been discovered that inventive vanadium-free catalysts with phosphorus, titanium, and optionally oxygen have shown surprising catalytic activity when used in the acetic acid and formaldehyde conversion to acrylic acid. In preferred embodiments, the surprising catalytic activity is demonstrated when the reaction is conducted at higher temperatures, e.g., greater than 320° C., greater than 340° C., or greater than 355° C.

Generally speaking, catalyst performance has been found to depend on the molar ratio of phosphorus to titanium. For example, without being bound by theory, it is believed that as the phosphorus-titanium ratio approaches the range of from 1.66 to 2.5, e.g., from 2.0 to 2.25, the acrylate product selectivity increases significantly. For example, depending on the temperature at which the acetic acid formation reaction is conducted, acrylate product selectivity is at least 40%, e.g., at least 50%, or at least 80%, when the phosphorus-titanium ratio ranges from 2.0 to 2.25.

The acrylates space time yield (“STY”) has been found to reach a maximum at phosphorus-titanium ratio at around 1.33 at lower reactor temperature, e.g., below 340° C. or below 320° C. At the lower temperature, the acrylate STY then decreases as the phosphorus-titanium ratio increases. In one embodiment, without being bound by theory, it is believed that this drop in acrylate STY is due to a loss of catalyst activity at the higher phosphorus-titanium ratio, which results in lower acetic acid and/or formaldehyde conversions.

At lower reactor temperature, e.g., 340° C., as phosphorus-titanium ratios increase past 1.33, acrylate STY actually decreases, e.g., acrylate STY is less than 20 grams/liter of catalyst/hour. Surprisingly and unexpectedly, when the acrylic acid formation reaction is conducted at higher temperatures, the acrylates STY continues to increase as the ratio between phosphorus and titanium increases. For example, at reactor temperature 355° C. and as phosphorus-titanium ratios increase pass 1.33, the acrylates STY is at least 20 grams/liter of catalyst/hour, e.g., at least 25 grams/liter of catalyst/hour, or at least 40 grams/liter of catalyst/hour. For example, at reactor temperature 370° C. and as the ratio between phosphorus and titanium increases pass 1.33, the acrylates STY is at least 20 grams/liter of catalyst/hour, e.g., at least 30 grams/liter of catalyst/hour, or at least 50 grams/liter of catalyst/hour.

Preferred embodiments of the inventive process also have low selectivity to undesirable products, such as carbon monoxide and carbon dioxide. The selectivity to these undesirable products preferably is less than 29%, e.g., less than 25% or less than 15%. More preferably, these undesirable products are not detectable. Formation of alkanes, e.g., ethane, may be low, and ideally less than 2%, less than 1%, or less than 0.5% of the acetic acid passed over the catalyst is converted to alkanes, which have little value other than as fuel.

The alkanoic acid or ester thereof and alkylenating agent may be fed independently or after prior mixing to a reactor containing the catalyst. The reactor may be any suitable reactor. Preferably, the reactor is a fixed bed reactor, but other reactors such as a continuous stirred tank reactor or a fluidized bed reactor, may be used.

In some embodiments, the alkanoic acid, e.g., acetic acid, and the alkylenating agent, e.g., formaldehyde, are fed to the reactor at a molar ratio of at least 0.10:1, e.g., at least 0.75:1 or at least 1:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, acrylate selectivity may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4100 kPa, e.g., from 3 kPa to 345 kPa, or from 6 to 103 kPa.

Water may be present in amounts up to 60 wt. %, by weight of the reaction mixture, e.g., up to 50 wt. % or up to 40 wt. %. Water, however, is preferably reduced due to its negative effect on process rates and separation costs.

In one embodiment, an inert or reactive gas is supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors.

In one embodiment, the unreacted components such as the carboxylic acid and formaldehyde as well as the inert or reactive gases that remain are recycled to the reactor after sufficient separation from the desired product.

When the desired product is an unsaturated ester made by reacting an ester of an alkanoic acid ester with formaldehyde, the alcohol corresponding to the ester may also be fed to the reactor either with or separately to the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity.

### EXAMPLES

### Example

10 catalyst samples were prepared using the following general procedure. These catalyst samples have phosphorus-titanium ratios ranging from 0.00:1.00 to 2.5:1.0. The specific composition of the 10 catalyst samples are shown in Table 2.

Ti(OiPr)₄ (1.0 mol equivalent) was slowly added to an equal volume of 2-propanol. The diluted Ti(OiPr)₄ solution was slowly added to 150 ml deionized water. This suspension was stirred for one hour at room temperature. Phosphoric acid (0.0 to 2.5 mol equivalent) was slowly added to form a suspension of hydrous titanium pyrophosphate. The suspension was stirred for one hour at room temperature. The resultant material was dried with rotary evaporator set at 95° C. The tacky solid was further dried at 120° C. overnight to a solid consistency. The solid was calcined using the following profile:

i) drying with flowing air at 160° C. for 2 hours;

ii) drying with flowing air at 350° C. for 4 hours;

iii) drying under static air at 450° C. for six hours.

TABLE 2
Catalyst Samples

| | | Nominal Stoichiometric | | | | | |
|---|---|---|---|---|---|---|---|
| | P/Ti | formula | | | (wt %) | (wt %) | (wt %) |
| Sample | ratio | Ti | P | O | Ti | P | O |
| 1 | 0.00 | 1 | 0.0 | 2.0 | 60 | 0 | 40 |
| 2 | 0.33 | 1 | 0.3 | 2.8 | 46 | 10 | 44 |
| 3 | 0.66 | 1 | 0.7 | 3.7 | 38 | 16 | 46 |
| 4 | 1.00 | 1 | 1.0 | 4.5 | 32 | 21 | 48 |
| 5 | 1.33 | 1 | 1.3 | 5.3 | 28 | 24 | 49 |
| 6 | 1.66 | 1 | 1.7 | 6.2 | 24 | 26 | 50 |
| 7 | 2.00 | 1 | 2.0 | 7.0 | 22 | 28 | 51 |
| 8 | 2.13 | 1 | 2.1 | 7.5 | 21 | 28 | 51 |
| 9 | 2.25 | 1 | 2.3 | 7.9 | 20 | 29 | 52 |
| 10 | 2.50 | 1 | 2.5 | 8.8 | 18 | 29 | 53 |

A reaction feed comprising acetic acid (9.1%), formaldehyde (17.3%), methanol (6.7%), water (38%), oxygen (4.06%), and nitrogen (24.8%) was passed through a fixed bed reactor comprising the catalyst samples from Table 2. The reaction was conducted at three temperatures, 340° C., 355° C., and 370° C. Acrylic acid and methyl acrylate (collectively, “acrylates”) were produced. The conversions, selectivities, and space time yields are shown in Table 3.

TABLE 3
Acrylate Production

| | | | Acrylate | Acrylate Space Time |
|---|---|---|---|---|
| Reaction | Catalyst | P:Ti | Product | Yield, g/liter of |
| Temperature | Sample | ratio | Selectivity | catalyst/hr |
| 340° C. | 1 | 0.00:1.00 | 8.4 | 8.8 |
| 340° C. | 2 | 0.33:1.00 | 27.2 | 22.8 |
| 340° C. | 3 | 0.66:1.00 | 31.0 | 26.2 |
| 340° C. | 4 | 1.00:1.00 | 43.4 | 29.9 |
| 340° C. | 5 | 1.33:1.00 | 58.4 | 35.5 |
| 340° C. | 6 | 1.66:1.00 | 66.0 | 32.7 |
| 340° C. | 7 | 2.00:1.00 | 91.6 | 19.9 |
| 340° C. | 8 | 2.13:1.00 | 92.0 | 11.8 |
| 340° C. | 9 | 2.25:1.00 | 77.8 | 13.0 |
| 340° C. | 10 | 2.50:1.00 | 76.4 | 19.6 |
| 355° C. | 1 | 0.00:1.00 | 3.0 | 4.2 |
| 355° C. | 2 | 0.33:1.00 | 13.5 | 9.3 |
| 355° C. | 3 | 0.66:1.00 | 20.5 | 10.6 |
| 355° C. | 4 | 1.00:1.00 | 37.8 | 17.4 |
| 355° C. | 5 | 1.33:1.00 | 46.2 | 23.4 |
| 355° C. | 6 | 1.66:1.00 | 57.4 | 25.5 |
| 355° C. | 7 | 2.00:1.00 | 84.4 | 22.4 |
| 355° C. | 9 | 2.25:1.00 | 88.4 | 28.7 |
| 355° C. | 10 | 2.50:1.00 | 75.4 | 42.9 |
| 370° C. | 3 | 0.66:1.00 | 3.2 | 3.0 |
| 370° C. | 4 | 1.00:1.00 | 33.2 | 16.4 |
| 370° C. | 5 | 1.33:1.00 | 50.2 | 25.6 |
| 370° C. | 6 | 1.66:1.00 | 53.4 | 28.7 |
| 370° C. | 7 | 2.00:1.00 | 81.5 | 33.0 |
| 370° C. | 8 | 2.13:1.00 | 83.4 | 55.7 |
| 370° C. | 9 | 2.25:1.00 | 82.9 | 44.1 |
| 370° C. | 10 | 2.50:1.00 | 75.0 | 57.3 |

As shown in Table 3, the vanadium-free catalyst compositions surprisingly and unexpectedly show a strong dependent relationship with the phosphorus/titanium ratio. In particular, Table 3 shows that acrylates product selectivity reached a maximum when the ratio between phosphorus and titanium is between 2.0:1.0 to 2.25:1.0. This trend is consistent for all three reaction temperatures.

Also, as shown in Table 3, at lower temperatures, e.g., 340° C., acrylates STY reached a maximum when the phosphorus-titanium ratio was 1.33:1.00. The acrylates STY then decreased as the ratio of phosphorus-titanium increased.

Surprisingly and unexpectedly, the acrylates STY at higher temperatures, e.g., 355° C. and 370° C., continued to increase as phosphorus-titanium ratio increases. For example, at 355° C. the acrylates STY reached 42.9 grams/liter of catalyst/hour and at 370° C. the acrylates STY reached 57.3 grams/liter of catalyst/hour for a phosphorus-titanium catalyst with a phosphorus-titanium ratio of 2.5:1.0. At lower temperatures, the acrylate STY reached a maximum value of 35.5 grams/liter of catalyst/hour at a phosphorus-titanium ratio of 1.33:1.00. Such a significant increase in acrylate STY at higher temperatures and the fact that acrylate STY continued to increase as phosphorus-titanium ratio increased are surprising and unexpected.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A catalyst composition, comprising:
from 18 wt. % to 35 wt. % phosphorus;
from 11 wt. % to 39 wt. % titanium; and
less than 1 wt. % vanadium, wherein the molar ratio of phosphorus to titanium is at least 1:1.

2. The catalyst composition of claim 1, wherein the catalyst is substantially free of vanadium.

3. The composition of claim 1, wherein the molar ratio of phosphorus to titanium is at least 2:1.

4. The composition of claim 1, wherein the molar ratio of phosphorus to titanium is at least 2.25:1.

5. The composition of claim 1, comprises from 23 wt. % to 30 wt. % of phosphorus and 15 wt. % to 36 wt. % of titanium.

6. The composition of claim 1, wherein the catalyst further comprises from 30 wt. % to 65 wt. % oxygen.

7. The catalyst composition of claim 1, wherein the catalyst comprises at least 15 wt. % titanium.

8. The catalyst composition of claim 1, wherein the catalyst comprises at least 18 wt. % phosphorus.

9. The catalyst composition of claim 1, wherein the catalyst further comprises a support.

10. The catalyst composition of claim 9, wherein the support is selected from the group consisting of silica, alumina, zirconia, titania, aluminosilicates, zeolitic materials, and mixtures thereof.

11. The catalyst composition of claim 1, wherein the catalyst is heterogeneous.

12. The catalyst composition of claim 1, wherein the catalyst is homogeneous.

13. The catalyst composition of claim 1, wherein the catalyst further comprises a binder.

14. The catalyst composition of claim 1, wherein the catalyst corresponds to the formula
TiₐP_{b}O_{c}
wherein the ratio of a to b is greater than 1:0.1.

15. The catalyst composition of claim 14, wherein:
a is 1;
b is from 1 to 2.5; and
c is from 2.25 to 9.

16. A process for producing a catalyst composition, comprising:
contacting a titanium precursor mixture with phosphoric acid to form a catalyst precursor mixture; and
calcining the catalyst precursor mixture to form the catalyst composition, comprising
from 18 wt. % to 35 wt. % phosphorus;
from 11 wt. % to 39 wt. % titanium; and
less than 1 wt. % vanadium, wherein the molar ratio of phosphorus to titanium is at least 1:1.

17. The process of claim 16, wherein the titanium precursor is selected from a group consisting of Ti(OR)₄, LₓTi(OR)_{y} complexes, TiCl_{z}, hydrated titania sols and colloidal TiO₂, wherein:
x ranges from 1 to 3,
y ranges from 1 to 3, and
z ranges from 3 to 4.

18. The process of claim 17, wherein L is a bidentate ligand.

19. The process of claim 17, wherein R is selected from the group consisting of methyl, ethyl, propyl and butyl.

20. A catalyst composition produced by the process of claim 16.

21. The process of claim 16, wherein the calcining comprises:
contacting the catalyst precursor mixture with flowing air at a first temperature;
contacting the catalyst precursor mixture with flowing air at a second temperature greater than the first temperature; and
contacting the catalyst precursor mixture with static air at a third temperature greater than the first and second temperature.

22. The process of claim 21, wherein the first temperature ranges from 110° C. to 210° C., the second temperature ranges from 300° C. to 400° C. and the third temperature ranges from 400° C. to 500° C.

23. A process for producing unsaturated acid and/or acrylate comprising the steps of:
contacting an alkanoic acid and an alkylenating agent over a catalyst, the catalyst comprising:
from 18 wt. % to 35 wt. % phosphorus;
from 11 wt. % to 39 wt. % titanium; and
less than 1 wt. % vanadium, wherein the molar ratio of phosphorus to titanium is at least 1:1, to produce the acrylic acid and/or acrylate.

24. The process of claim 23, wherein the alkylenating agent comprises formaldehyde.

25. The process of claim 23, wherein the contacting is conducted at a temperature greater than 355° C.

26. The process of claim 23, wherein the contacting is conducted at a temperature greater than 340° C. and the molar ratio of phosphorus to titanium is greater than 1.25:1.0.

27. The process of claim 23, wherein the contacting is conducted at a temperature greater than 355° C. and the molar ratio of phosphorus to titanium is greater than 2.0:1.0.

28. The process of claim 23, wherein the contacting is conducted at a temperature greater than 370° C. and the molar ratio of phosphorus to titanium is greater than 2.0:1.0.

29. The process of claim 23, wherein the product selectivity of acrylates is at least 31% when the contacting is conducted at 340° C.

30. The process of claim 23, wherein the product selectivity of acrylates is at least 21% when the contacting is conducted at 355° C.

31. The process of claim 23, wherein the product selectivity of acrylates is at least 4% when the contacting is conducted at 370° C.
